SEQUENCE LISTING (21 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 30 aa, Homo sapiens
FVNQHLCGSHLVEALYLVCGERGFFYTKPT

SEQ ID NO: 5 - protein, 30 aa, Homo sapiens
FVNQHLCGSHLVEALYLVCGERGFFYTDKT

SEQ ID NO: 6 - protein, 30 aa, Homo sapiens
FVNQHLCGSDLVEALYLVCGERGFFYTPKT

SEQ ID NO: 7 - protein, 30 aa, Homo sapiens
FVNQHLCGSDLVEALYLVCGERGFFYTKPT

SEQ ID NO: 8 - protein, 21 aa, Homo sapiens
  MISC_FEATURE (1)..(1): Xaa=D-Ala, D-Leu, or D-Asp, Phe
XIVEQCCXSICSLYQLENYCN

SEQ ID NO: 9 - protein, 21 aa, Homo sapiens
  MISC_FEATURE (8)..(8): Xaa=Dbu
FIVEQCCXSICSLYQLENYCN

SEQ ID NO: 10 - protein, 21 aa, Homo sapiens
  MISC_FEATURE (1)..(1): Xaa=D-Ala
XIVEQCCXSICSLYQLENYCN

SEQ ID NO: 11 - protein, 21 aa, Homo sapiens
  MISC_FEATURE (1)..(1): Xaa=D-Leu
XIVEQCCXSICSLYQLENYCN

SEQ ID NO: 12 - protein, 21 aa, Homo sapiens
  MISC_FEATURE (1)..(1): Xaa=D-Asp
XIVEQCCXSICSLYQLENYCN

SEQ ID NO: 13 - protein, 22 aa, Homo sapiens
RGIVEQCCRSICSLYQLENYCG

SEQ ID NO: 14 - protein, 22 aa, Homo sapiens
RGIVEQCCHSICSLYQLENYCG

SEQ ID NO: 15 - protein, 21 aa, Homo sapiens
GIVEQCCHSICSLYQLENYCG

SEQ ID NO: 16 - protein, 21 aa, Homo sapiens
GIVAQCCHSICSLYQLENYCN

SEQ ID NO: 17 - protein, 21 aa, Homo sapiens
GIVHQCCHSICSLYQLENYCN

SEQ ID NO: 18 - protein, 21 aa, Homo sapiens
GIVAQCCHSICSLYQLENYCG

SEQ ID NO: 19 - protein, 30 aa, Homo sapiens
HVNQHLCGSHLVEALYLVCGERGFFYTPKT

SEQ ID NO: 20 - protein, 21 aa, Homo sapiens
GIVHQCCTSICSLYQLENYCN

SEQ ID NO: 21 - protein, 21 aa, Homo sapiens
GIVEQCCHSICSLYQLENYCN

(19) United States
(12) Patent Application Publication
(10) Pub. No.: US 2012/0184488 A1
(43) Pub. Date: Jul. 19, 2012

(54) INSULIN ANALOGUES OF ENHANCED RECEPTOR-BINDING SPECIFICITY

(75) Inventor: Michael Weiss, Moreland Hills, OH (US)

(73) Assignee: CASE WESTERN RESERVE UNIVERSITY, Cleveland, OH (US)

(21) Appl. No.: 13/393,745

(22) PCT Filed: Sep. 1, 2010
(86) PCT No.: PCT/US10/47546

§ 371 Date: Apr. 4, 2012

Related U.S. Application Data
(60) Provisional application No. 61/238,871, filed on Sep. 1, 2009.

(51) Int. Cl. A61K 38/28; C07K 14/62; A61P 3/10

(52) U.S. Cl. 514/6.2; 530/303

(57) ABSTRACT

A method of treating a patient includes administering a physiologically effective amount of an insulin analogue or a physiologically acceptable salt thereof to the patient. The insulin analogue or physiologically acceptable salt thereof contains an insulin A-chain sequence modified at positions selected from the group consisting of A0, A1, A4, A8, and A21. The insulin analogue may exhibit decreased affinity for the IGF receptor in comparison to wild type insulin of the same species and at least 20% of the affinity of wild-type insulin for the insulin receptor of the same species. Position A0 may be arginine. Position A1 may be D-alanine, D-aspartic acid, or D-leucine. Position A8 may be histidine, lysine, or arginine. Optionally, an insulin B-chain analogue sequence comprises a histidine at position B1. A nucleic acid may encode such an insulin polypeptide.

### CROSS REFERENCE TO RELATED APPLICATIONS

This application claims priority from PCT/US2010/047546 filed on Sep. 1, 2010, which claims priority from U.S. Provisional Patent Application No. 61/238,871 filed on Sep. 1, 2009.

### STATEMENT REGARDING FEDERALLY SPONSORED RESEARCH OR DEVELOPMENT

This invention was made with government support under cooperative agreements awarded by the National Institutes of Health, Contract Nos. NIH RO1DK40949, RO1DK069764 and R01-DK74176. The U.S. government may have certain rights to the invention.

### INCORPORATION BY REFERENCE OF MATERIAL SUBMITTED ELECTRONICALLY

The material contained in the Sequence Listing provided herewith in ASCII compliant format in the text file entitled “200512-145_ST25.txt” created on Mar. 1, 2012 and containing 7220 bytes, is hereby incorporated by reference herein.

### BACKGROUND OF THE INVENTION

The functional specificity of proteins used in medical treatment is an important concern in medicine. Binding of protein hormones and growth factors to cognate cellular receptors mediates biological effects. Cross-binding of such protein hormones and growth factors to other receptors can lead to undesired biological effects of treatment. Insulin and insulin analogues regulate metabolism through binding to the insulin receptor (IR) and can mediate undesirable mitogenic effects through binding to the Type I insulin-like growth factor receptor (IGFR). The present invention pertains to the functional specificity of insulin and insulin analogues. More particularly, the present invention discloses amino-acid substitutions in the insulin molecule that decrease the binding of insulin analogues to IGFR and enhance the ratio of the affinity of the hormone for IR to its affinity for IGFR.

Administration of insulin is long established as a treatment for diabetes mellitus. Insulin is a small globular protein that plays a central role in metabolism in vertebrates. Insulin contains two chains, an A-chain, containing 21 residues and a B-chain containing 30 residues. The hormone is stored in the pancreatic β-cell as a Zn²⁺-stabilized hexamer, but functions as a Zn²⁺-free monomer in the bloodstream. Insulin is the product of a single-chain precursor, proinsulin, in which a connecting region (35 residues) links the C-terminal residue of B-chain (residue B30) to the N-terminal residue of the A-chain (FIG. 1A). The structure of proinsulin, as recently determined by nuclear magnetic resonance as an engineered monomer, contains an insulin-like core and disordered connecting peptide, as previously hypothesized (FIG. 1B). Formation of three specific disulfide bridges (A6-A11, A7-B7, and A20-B19; FIG. 1B) is thought to be coupled to oxidative folding of proinsulin in the rough endoplasmic reticulum (ER). Proinsulin assembles to form soluble Zn²⁺-coordinated hexamers shortly after export from ER to the Golgi apparatus. Endoproteolytic digestion and conversion to insulin occurs in immature secretory granules followed by morphological condensation. Crystalline arrays of zinc insulin hexamers within mature storage granules have been visualized by electron microscopy (EM). Assembly and disassembly of native oligomers is thus intrinsic to the pathway of insulin biosynthesis, storage, secretion, and action.

Insulin belongs to a superfamily of vertebrate insulin-related proteins, including (in addition to insulin itself) insulin-related growth factors I and II (IGF-I and IGF-II), relaxin, and relaxin-related factors. These proteins exhibit homologous α-helical domains and disulfide bridges. IGFs are single-chain polypeptides containing A- and B domains, an intervening connecting (C) domain, and C-terminal D domain; due to proteolytic processing insulin and relaxin-related factors contain two chains (designated A and B). Whereas the six motif-specific cysteines and selected core residues are broadly conserved throughout the vertebrate insulin-related superfamily, other residues are restricted to particular proteins, giving rise to functional specificity. Insulin and IGFs function as ligands for receptor tyrosine kinases (the insulin receptor (IR) and class I IGF receptor (IGFR)), whereas relaxin and related factors bind to G-protein coupled receptors (GPCRs). Insulin binds most strongly to IR, weakly to IGFR, and is without detectable binding to GPCRs. IGF-I binds most strongly to IGFR, weakly to IR, and is without detectable binding to GPCRs. Cross-binding of insulin to IGFR can trigger mitogenic signaling pathways, including those associated with proliferation of cancer cells. The long-term safety of insulin replacement therapy in the treatment of diabetes mellitus may be enhanced by use of insulin analogues containing amino-acid substitutions that decrease the extent of such cross-binding. Such amino-acid substitutions would enhance the ratio of affinity of the insulin analogue for IR versus IGFR.

The majority of insulin products in current use for the treatment of diabetes mellitus contain insulin analogues whose sequence differs from that of natural human insulin. Amino-acid substitutions in the A- and/or B-chains of insulin have widely been investigated for possible favorable effects on the pharmacokinetics of insulin action following subcutaneous injection. Examples are known in the art of substitutions that accelerate or delay the time course of absorption. The former analogues collectively define the “meal-time” insulin analogues because patients with diabetes mellitus may inject such rapid-acting formulations at the time of a meal whereas the delayed absorption of wild-type human insulin or animal insulins (such as porcine insulin or bovine insulin) makes it necessary to inject these formulations 30-45 minutes prior to a meal. The substitutions are designed to destabilize the zinc insulin hexamer by altering the steric or electrostatic complementarity of subunit interfaces and thereby to facilitate the rapid dissociation of the zinc insulin hexamer after subcutaneous administration. Also known in the art are long-acting insulin analogues whose slow absorption over 12-24 hours provides basal control of blood glucose concentrations. Such analogues, exemplified but not restricted to [Gly^{A21}, Arg^{B31}, Arg^{B32}]-insulin (insulin glargine or Lantus®), may contain amino-acid substitutions and/or extensions of the A- or B-chains designed to shift the isoelectric point of the insulin analogue upward toward neutrality. The analogues are typically formulated as soluble insulin monomers, dimers, and higher-order oligomers at pH<5 under which conditions zinc-mediated assembly is impaired by protonation of His^{B10}. Prolonged absorption is achieved by aggregation and precipitation of the insulin analogue in the subcutaneous tissue due to a shift in pH toward 7.4. Another type of long-acting insulin analogue is exemplified by insulin Detimir® in which residue Thr^{B30} has been deleted and a C14 fatty acid chain is connected to the side chain of Lys^{B29}. The fatty acid chain mediates binding of the insulin analogue to serum albumin and hence extends its circulating lifetime.

The biological, physical, and chemical properties of insulin analogues can be altered relative to human insulin due to the presence of amino-acid substitutions in the A-chain and/or B-chain or due to possible extensions of the A-chain and/or B-chain to create a larger molecule. Studies of insulin analogues have indicated that the properties of analogues containing two or more modifications cannot reliably be predicted based on the properties of a set of analogues containing corresponding single modifications. Because an amino-acid substitution or chain extension at one location in the molecule can lead to transmitted changes in the conformation, dynamics, or solvation of the protein, effects of an amino-acid substitution at another location in the molecule can differ from the effects of the same substitution in the absence of the first modification. An example of an unanticipated transmitted effect of a modification is provided by distortions in the crystal structure of Arg^{A0}-insulin, which have been associated with decreased receptor binding. N-terminal extension of the A-chain to include Arg^{A0} thus alters the structural environments of residues A4, A8, and other sites. Amino-acid substitutions or chain extensions that insert one or more basic residues (Arg or Lys) in general result in an upward shift in the isoelectric point toward neutrality; the extent of this shift is influenced by the structure, solvation, and transmitted conformational changes associated with the modification, and so experience has taught that observed pIs are not well predicted by the properties of the isolated amino acids. While not wishing to be restrained by theory, experience has taught that the combined effects of two or more modifications can be unanticipated based on the properties of analogues containing single modifications. It is therefore possible that novel combinations of modifications may together have properties that provide unique advantages for the therapeutic use of insulin analogues in the treatment of diabetes mellitus.

Concern for the safety of insulin analogues that exhibit increased relative or absolute affinity for IGFR was first raised more than ten years ago by the enhanced mitogenicity of Asp^{B10}-insulin in cell culture studies of human cancer cell lines (relative to human insulin) and by an increased incidence of mammary carcinomas in Sprague-Dawley rats being treated by Asp^{B10}-insulin (relative to treatment with human insulin). Asp^{B10}-insulin was accordingly not pursued as a clinical insulin analogue formulation for human use. In the past year analogous concerns have been raised regarding Lantus®, which also exhibits enhanced cross-binding to IGFR and increased mitogenicity in human cell culture. A recent retrospective case study of more than 120,000 European patients with diabetes mellitus being treated with Lantus® suggested a dose-dependent increase in the incidence of diverse cancers, including cancers of the breast, prostate, colon, and pancreas. The extent of cancer risk may be increased not only by the elevated level of cross-binding to IGFR, but the reduced affinity of Lantus® for IR, which makes necessary the injection of more micrograms of insulin analogue per unit of hypoglycemic activity.

Human insulin itself can bind to IGFR but with an affinity 333-fold lower than that of its binding to IR. Meal-time insulin analogues such as Humalog® and Novolog® exhibit a similar level of cross-binding to IGFR (the cross-binding of insulin lispro (the active component of Humalog®) has been reported to be slightly increased). Epidemiological studies have revealed an association between endogenous hyperinsulinemia (a compensatory response to insulin resistance in the metabolic syndrome and early stages of type 2 diabetes mellitus) with increased prevalence of cancer, especially colorectal cancer. Treatment of patients with insulin resistance with human insulin or insulin analogues at high doses may also be associated with an increase in cancer risk, which may reflect this baseline level of cross-binding to IGFR. For such patients it is possible that even the baseline receptor specificity of human insulin and meal-time insulin analogues may be insufficiently stringent to ensure the safety of long-term treatment with respect to cumulative cancer risk.

Regulation of blood glucose concentrations by insulin analogues does not require binding to IR at the precise level of human insulin. A decrease in the affinity of an analogue for IR can be compensated in vivo by a delay in its clearance from the bloodstream. Such compensation occurs because clearance of insulin is mediated by its binding to IR. Insulin analogues with threefold reduced affinity for IR can nonetheless exhibit in vivo potencies similar to that of human insulin. Further decreases in affinity can be compensated by increase in the amount of analogue injected. Examples of insulin analogues with such decreased affinity are Lantus® and insulin Detemir®. Changes in the affinity of insulin analogues for IR usually reflect changes in off rates: reductions in affinity are associated with shortening of the residence time of the hormone on the receptor whereas increases in affinity are associated with prolongation of the residence time. It is not known what in general are the relationships between residence time and metabolic potency or between residence time and mitogenic signaling. Prolonged residence time of Asp^{B10}-insulin on the IR complex has been proposed to underlie, at least in part, its enhanced mitogenicity. While not wishing to be constrained by theory, past experience has taught that insulin analogues with relative in vitro affinities for IR between 20% and 200% relative to human insulin can be effective for the treatment of diabetes mellitus in mammals.

Therefore, there is a need for insulin analogues that exhibit reduced cross-binding to IGFR while maintaining at least a portion of the biological activity of the analogue in control of blood glucose concentration. In particular, there is a need for insulin analogues that exhibit self-assembly and solubility properties similar to that of human insulin but with decreased IGFR affinity while maintaining at least a portion of the biological activity of the analogue in control of blood glucose concentration. There is a further need for insulin analogues that exhibit an increase in isoelectric point toward neutrality without increase in IGFR affinity while maintaining at least a portion of the biological activity of the analogue in control of blood glucose concentration.

### SUMMARY OF THE INVENTION

The present invention provides insulin analogues containing either an amino acid addition at position A0 (that is, an addition at the amino terminal end of the A-chain) or amino-acid substitutions at positions A4, A8, or A21 of insulin or combinations thereof. Without wishing to condition patentability on any particular theory, side chains at these sites are each believed to project into solvent from the surface of the A-chain in both an insulin monomer and on its assembly into an insulin hexamer, thus enabling diverse side chains to be accommodated without steric interference but in combination providing favorable biological, chemical, and/or physical properties.

In the native structure of insulin, residues A1-A8 comprise an α-helix. This segment is thought to contribute to the binding of insulin and insulin analogues to both IR and IGFR. While not wishing to condition patentability on theory, it is believed that invariant residues Ile^{A2} and Val^{A3} (shared between insulin and IGF-I) are required for binding of insulin analogues to either receptor and that the pro-L H_{α} atom of Gly^{A1} (also shared between insulin and IGF-I) is required for binding of insulin analogues to either receptor. Additionally, invariant cysteines at positions A6 and A7 (broadly conserved as framework disulfide bridges characteristic of insulin and insulin-related polypeptides) are likewise thought to be required for binding of insulin analogues to either receptor. Therefore, modification at these sites was avoided in the present invention. However, single amino-acid substitutions at A0, A4, and A8 are known to be possible with maintenance of at least 50% affinity for the insulin receptor relative to wild-type human insulin. Substitution of Asn^{A21} by Gly is known in the art to retard the chemical degradation of insulin analogues when formulated under acidic conditions.

It is, therefore, desired to provide insulin analogues that are retain high affinity for the insulin receptor with decreased cross-binding to the Type I IGF receptor.

The A1-A8 α-helix of insulin or of insulin analogues contributes to its isoelectric point (pI) by its combination of charged sites, neutral sites, α-helical dipole moment, and mutual electrostatic interactions. While again not wishing to be constrained by theory, an upward shift in pI would be anticipated by addition of a basic residue (Arg or Lys), addition of His (whose imidazole ring may be protonated to bear a positive charge), or removal of an acidic residue. A basic residue is observed in human insulin at position A4. A neutral residue at position A8 may be replaced by His or a basic residue. N-terminal extension of the A-chain by one or two basic residues is known in the art to cause an upward shift in pI toward neutrality.

It is therefore also desirable to provide insulin analogues that exhibit the above receptor-binding properties and also exhibit an upward shift in isoelectric point toward neutrality.

In general, a method of treating a patient comprises administering a physiologically effective amount of an insulin analogue or a physiologically acceptable salt thereof to the patient, where the analogue or a physiologically acceptable salt thereof contains an insulin A-chain sequence modified at positions A0, A1, A4, A8, and/or A21 or a subset thereof. In one example, the A0 extension is Arg, the A8 substitution is Arg, and the A21 substitution is Gly. In another example, the A0 extension is Arg, the A8 substitution is His, and the A21 substitution is Gly. In another example, the A4 substitution is His or Ala and the A8 substitution is His. In yet another example the A1 substitution is a D-amino acid and the A8 substitution is di-amino-butyric acid.

An insulin analogue may be an analogue of any vertebrate insulin or insulin analogue containing a modified B-chain known in the art to confer altered absorption after subcutaneous injection. In one example, the insulin analogue is a mammalian insulin analogue such as human, murine, bovine, equine, or canine insulin analogues. In other examples, the insulin analogue is an analogue of sheep, whale, rat, elephant, guinea pig or chinchilla insulin.

Specific insulin analogues include those containing SEQ. ID. NOS. 9-21. A nucleic acid may encode a polypeptide having one of these sequences. Such a nucleic acid may be part of an expression vector, which may be used to transform a host cell.

### BRIEF DESCRIPTION OF THE SEVERAL VIEWS OF THE DRAWINGS

FIG. 1A is a schematic representation of the sequence of human proinsulin including the A- and B-chains and the connecting region shown with flanking dibasic cleavage sites (filled circles) and C-peptide (open circles).

FIG. 1B provides a structural model of proinsulin, consisting of an insulin-like moiety and disordered connecting peptide (dashed line).

FIG. 1C provides a representation of a proposed pathway of insulin fibrillation via partial unfolding of monomer. The native state is protected by classic self-assembly (far left). Disassembly leads to equilibrium between native- and partially folded monomers (open triangle and trapezoid, respectively). This partial fold may unfold completely as an off-pathway event (open circle) or aggregate to form a nucleus en route to a protofilament (far right).

FIG. 1D is a schematic representation of the sequence of human insulin indicating the position of residue A8 in the A-chain and sites of substitution in the B-chain known in the art to confer rapid absorption after subcutaneous injection.

FIG. 2 is a graph illustrating receptor-binding studies of insulin analogues containing D-substitutions at position A1 and di-amino-butyric acid at position A8. (A) Binding of insulin analogs to the IR (isoform B). (B) Cross-binding of insulin analogs to IGFR. Data represent competitive displacement of ¹²⁵I-labeled insulin (A) or ¹²⁵I-labeled IGF-1 (B). Symbol code in each panel: (♦) wild-type insulin, (▾) Dab^{A8}-insulin, (▪) [D-Ala^{A1}, Dab^{A8}]-insulin, and (▴) [D-Leu^{A1}, Dab^{A8}]-insulin; for clarity data points of [D-Asp^{A1}, Dab^{A8}]-insulin are not shown.

FIG. 3 is a graph illustrating receptor-binding studies of Arg^{A0}-His^{A8}-Gly^{A21}-insulin in relation to wild-type human insulin and insulin glargine (Lantus®). The vertical axis represents the 100 times the ratio of bound to total tracer ligand (B/Bₒ); the horizontal axis represents the logarithm (base 10) of the insulin analogue concentration (in nM) as in FIG. 2. (A) Binding of insulin analogs to the IR (isoform B). (B) Cross-binding of insulin analogs to IGFR. Data represent competitive displacement of ¹²⁵I-labeled insulin (A) or ¹²⁵I-labeled IGF-1 (B). Symbol code in each panel: (▪) wild-type insulin, (▾) insulin glargine, (▴) Arg^{A0}-His^{A8}-Gly^{A21}-insulin, and () human IGF-I.

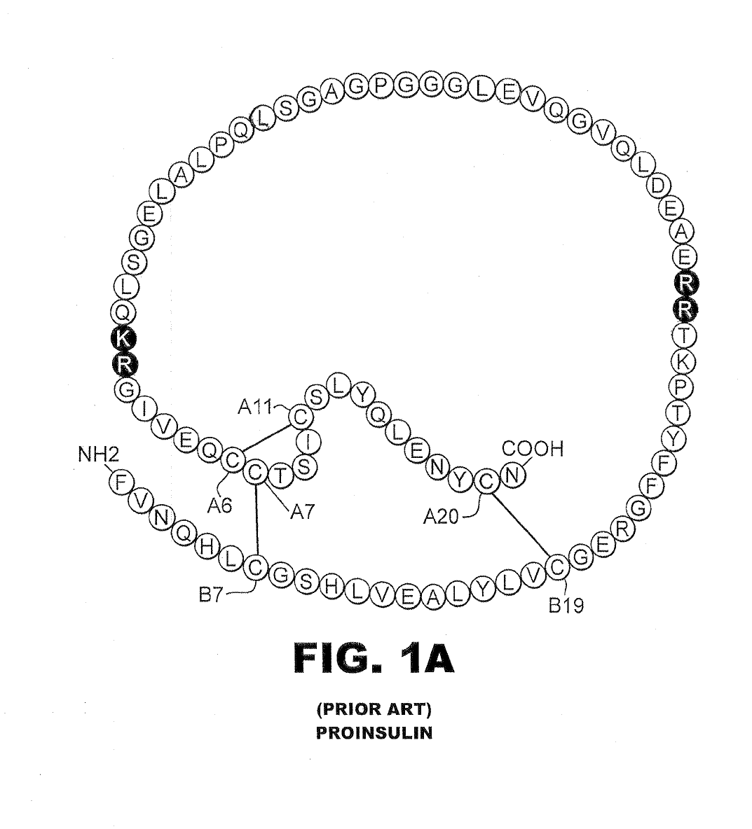

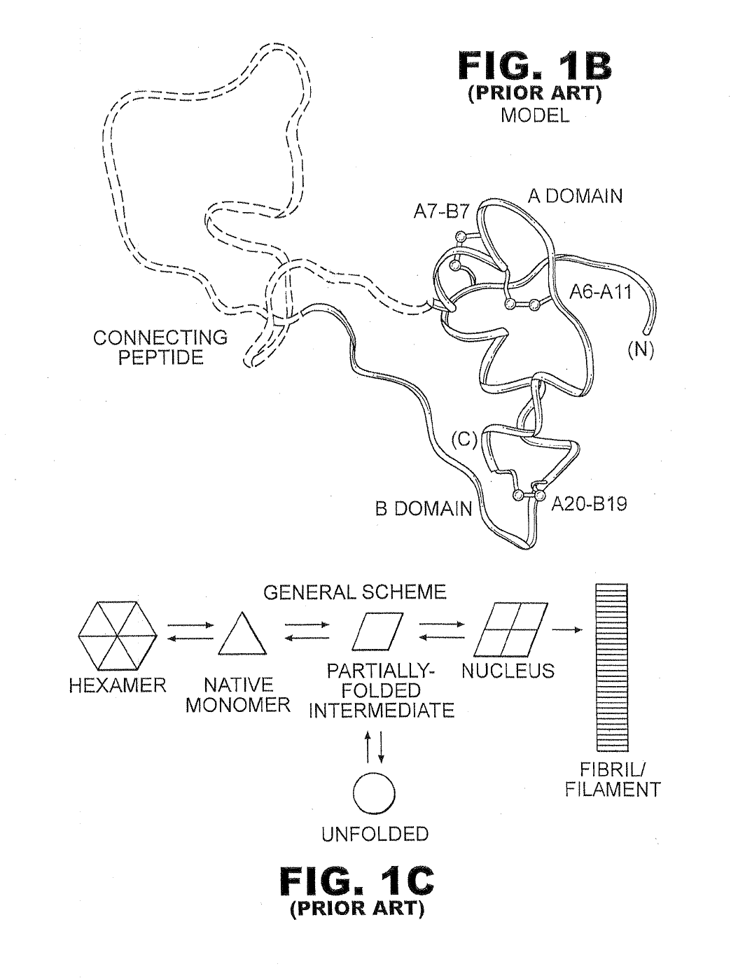

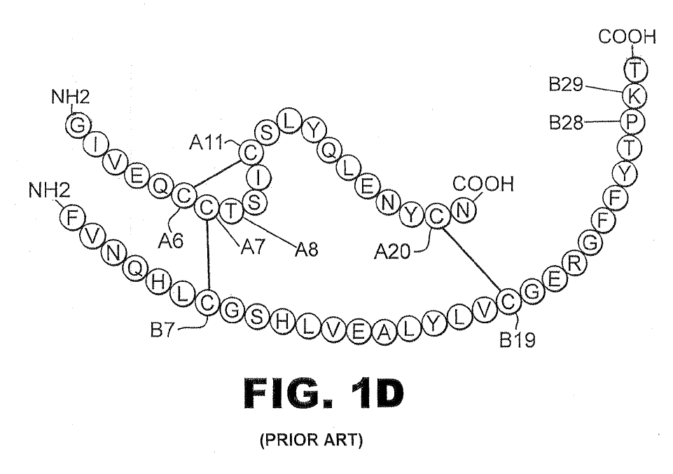

### DETAILED DESCRIPTION OF THE INVENTION

The present invention is directed toward decreasing the absolute and relative binding of insulin analogues to the Type 1 IGF receptor facilitate their use in treatment of diabetes, particularly with respect to the risk of cancer. To that end, the present invention provides insulin analogues that contain an amino-acid addition at position A0 and/or substitutions at positions A4, A8 and/or A21 of the A-chain polypeptide, or a subset thereof, and an insulin B-chain polypeptide, optionally containing one or more substitutions to confer rapid absorption following subcutaneous injection.

The insulin analogues of the present invention may also contain other modifications. As used in this specification and the claims, various substitutions in analogues of insulin may be noted by the convention that indicates the amino acid being substituted, followed by the position of the amino acid, optionally in superscript. The position of the amino acid in question includes the A- or B-chain of insulin where the substitution is located. For example, an insulin analogue of the present invention may also contain a substitution of Aspartic acid (Asp or D) or Lysine (Lys or K) for Proline (Pro or P) at amino acid 28 of the B-chain (B28), or a substitution of Pro for Lys at amino acid 29 of the B-chain (B29) or a combination thereof. These substitutions may also be denoted as Asp^{B28}, Lys^{B28}, and Pro^{B29}, respectively. Similarly, an insulin of the present invention may contain a substitution of Arginine (Arg or R), Histidine (His or H), or Lysine (Lys or K) at amino acid 8 of the A-chain (A8) in place of Threonine (Thr or T). These substitutions may be denoted Arg^{A8}, His^{A8}, or Lys^{A8}, respectively. Unless noted otherwise or wherever obvious from the context, the amino acids noted herein should be considered to be L-amino acids. D-amino acids are noted with the letter “D” as a prefix to the standard one- or three-letter abbreviation for the amino acid in question. For example, D-Alanine is abbreviated “D-Ala,” D-Leucine is abbreviated “D-Leu,” and D-Aspartate is abbreviated “D-Asp.”

The invention provides insulin analogues that exhibit reduced affinity for IGFR while retaining at least a portion of their affinity for IR and hence biological activity.

It is envisioned that other amino-acid substitutions at position A8 may also confer improved thermodynamic stability and resistance to fibrillation of an insulin analogue (relative to a corresponding analogue containing Thr^{A8}) while maintaining at least a majority of the activity of the analogue.

A method for treating a patient comprises administering an insulin analogue to the patient. In one example, the insulin analogue is an insulin analogue containing modifications in the A-chain that reduce its affinity for IGFR. In another example, the insulin analogue also exhibits an upward shift in isoelectric point (pI) toward neutrality. The insulin analogue is administered by subcutaneous injection, by an external insulin pump, or by an implantable insulin pump.

It is also envisioned that it would be possible to apply the introduction of substitutions at position A8 to long-acting analogs formulated at acidic pH and designed to exhibit a higher isoelectric point than wild-type human insulin for one or all of the purposes of decreasing the solubility of the insulin analog in the subcutaneous depot and reducing a possible cancer risk proposed to be associated with cross-binding of insulin and insulin analogues to the Type 1 IGF receptor.

It is further envisioned that it would be possible to apply the introduction of combined substitutions at positions A1, A4 and/or A8, or a subset thereof, in other classes of formulations of insulin analogues (such as but not restricted to regular, NPH, semi-lente and lente, including mixtures of such types) for one or more of the purposes of decreasing cross-binding of such analogues to the Type 1 IGF receptor.

It has been discovered that substitutions at position A8 in concert with either substitutions at position A4 or N-terminal extension of the A-chain by a basic amino acid can reduce cross-binding by an insulin analogue to the Type I IGF receptor and effect an upward shift in pI toward neutality.

It has been discovered that substitution at position A8 by a non-standard basic side chain (di-amino-butryic acid, for example) in concert with D-amino-acid substitutions at position A1 can reduce cross-binding by an insulin analogue to the Type I IGF receptor.

In general, a vertebrate insulin analogue or a physiologically acceptable salt thereof, comprises a insulin analogue containing an insulin A-chain and an insulin B-chain. An insulin analogue of the present invention may also contain other modifications, such as substitutions of a basic amino-acid extensions of the B-chain at residues B1 and/or B31. In one example, the vertebrate insulin analogue is a mammalian insulin analogue, such as a human, porcine, bovine, feline, canine or equine insulin analogue. An insulin analogue of the present invention may also contain other modifications, such as a tether between the C-terminus of the B-chain and the N-terminus of the A-chain as described more fully in co-pending U.S. patent application Ser. No. 12/419,169, the disclosure of which is incorporated by reference herein.

A pharmaceutical composition may comprise such insulin analogues and which may optionally include zinc. Zinc ions may be included in such a composition at a level of a molar ratio of between 2.2 and 6 per hexamer of the insulin analogue. In such a formulation, the concentration of the insulin analogue would typically be between about 0.1 and about 3 mM. Modifications of meal-time insulin analogues may be formulated as described for (a) “regular” formulations of Humulin™ (Eli Lilly and Co.), Humalog™ (Eli Lilly and Co.), Novalin™ (Novo-Nordisk), and Novalog™ (Novo-Nordisk) and other rapid-acting insulin formulations currently approved for human use, (b) “NPH” formulations of the above and other insulin analogues, and (c) mixtures of such formulations.

Excipients may include glycerol, glycine, other buffers and salts, and anti-microbial preservatives such as phenol and meta-cresol; the latter preservatives are known to enhance the stability of the insulin hexamer. Such a pharmaceutical composition may be used to treat a patient having diabetes mellitus or other medical condition by administering a physiologically effective amount of the composition to the patient.

A nucleic acid comprising a sequence that encodes a polypeptide encoding an insulin analogue containing a sequence encoding an A-chain with a combination of an addition at position A0 and/or substitutions of at positions A4, A8 and/or A21 is also envisioned. The particular sequence may depend on the preferred codon usage of a species in which the nucleic acid sequence will be introduced. The nucleic acid may also encode other modifications of wild-type insulin. The nucleic acid sequence may encode a modified A- or B-chain sequence containing an unrelated substitution or extension elsewhere in the polypeptide or modified proinsulin analogues. The nucleic acid may also be a portion of an expression vector, and that vector may be inserted into a host cell such as a prokaryotic host cell like an E. coli cell line, or a eukaryotic cell line such as S. cereviciae or Pischia pastoris strain or cell line.

D-amino-acid substitutions at position A1, introducible by chemical synthesis, may modulate the isoelectric point of an insulin analogue. Adjusting the isoelectric point of an insulin analogue can be of pharmacological interest as a means to enhance or impair solubility at pH 7.4 and as a means to reduce the risk of unfavorable isoelectric precipitation of the protein during storage and use in an insulin pump. In particular, substitution of D-Asp or D-Glu at A1 can adjust downward the pI of an insulin analogue whereas substitution of D-Lys or D-Arg at A1 can adjust upward the pI of an insulin analogue. Non-standard amino acids with basic side chains may also be introduced at A1 or A8, such as ornithine (Orn), D-di-amino-butyric acid (Dbu), or D-di-amino-propionic acid (Dpr).

L-amino-acid substitutions at position A4 or A8, introducible by chemical synthesis or genetic engineering, may likewise modulate the isoelectric point of an insulin analogue. Because the naturally occurring residue at A4 has a negative charge, its substitution by an uncharged residue would be expected to shift the isoelectric point upward whereas its substitution by Arg, His, Lys or non-standard basic amino acid (ornithine, di-amino-butyric acid, or di-amino-propionic acid) would be expected to cause a larger such shift.

Substitutions with amino acids other than those containing nitrogenous side chains are also envisioned. For example, substitution of threonine by glutamate at A8 stabilizes insulin analogues. Although Glu^{A8} also reduces activity about 2-3 fold compared to Thr^{A8} insulin and insulin analogues, this level of activity may be acceptable for some applications. Additional A8 substitutions that are envisioned include arginine, phenylalanine, tyrosine and other non-beta-branched amino acid substitutions.

It is further envisioned that A8 substitutions can be designed to reduce cross-binding of insulin analogues to the IGF Type I receptor and hence the mitogenicity and potential carcinogenicity of the analogue.

Still other substitutions are also compatible with the insulin analogues of the present invention. In addition to the fast-acting class of insulin analogues, combinations of A0, A4, and A8 substitutions may be employed to decrease the IGFR cross-binding of long-acting insulin analogs and cause an addition upward shift in isoelectric point. An example is provided by (but not restricted to) insulin glargine (Lantus®), which is formulated at pH 4 but which undergoes aggregation in a subcutaneous depot at physiological pH. The substitutions of the present invention may also be combined with B-chain modifications that augment IGFR cross-binding to mitigate this unfavorable property; examples include extension of the B-chain by one or two basic residues (such as Arg^{B31}, Lys^{B31}, Arg^{B31}-Arg^{B32}, Arg^{B31}-Lys^{B32}, Lys^{B31}-Arg³², and Lys^{B31}-Lys^{B32}) or substitution of His^{B10} by Asp or Glu.

It is further envisioned that the insulin analogues of the present invention may also utilize any of a number of changes present in existing insulin analogues, modified insulins, or within various pharmaceutical formulations, such as regular insulin, NPH insulin, lente insulin or ultralente insulin, in addition to human insulin. The insulin analogues of the present invention may also contain substitutions present in analogues of human insulin that, while not clinically used, are still useful experimentally, such as DKP-insulin, which contains the substitutions Asp^{B10}, Lys^{B28} and Pro^{B29} or the Asp^{B9} substitution. The present invention is not, however, limited to human insulin and its analogues. It is also envisioned that these substitutions may also be made in animal insulins such as porcine, bovine, equine, and canine insulins, by way of non-limiting examples. Furthermore, in view of the similarity between human and animal insulins, and use in the past of animal insulins in human diabetic patients, it is also envisioned that other minor modifications in the sequence of insulin may be introduced, especially those substitutions considered “conservative” substitutions. For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine (Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E).

The amino acid sequence of human proinsulin is provided, for comparative purposes, as SEQ. ID. NO. 1. The amino acid sequence of the A-chain of human insulin is provided as SEQ. ID. NO. 2. The amino acid sequence of the B-chain of human insulin is provided, for comparative purposes, as SEQ. ID. NO. 3.

SEQ. ID. NO. 1 (proinsulin)
Phe-Val-Asn-Gln-His-Leu-Cys-Gly-Ser-His-Leu-Val-Glu-Ala-Leu-Tyr-Leu-Val-Cys-Gly-Glu-
Arg-Gly-Phe-Phe-Tyr-Thr-Pro-Lys-Thr-Arg-Arg-Glu-Ala-Glu-Asp-Leu-Gln-Val-Gly-Gln-Val-
Glu-Leu-Gly-Gly-Gly-Pro-Gly-Ala-Gly-S er-Leu-Gln-Pro-Leu-Ala-Leu-Glu-Gly-Ser-Leu-Gln-
Lys-Arg-Gly-Ile-Val-Glu-Gln-Cys-Cys-Thr-Ser-Ile-Cys -S er-Leu-Tyr-Gln-Leu-Glu-Asn-Tyr-
Cys-Asn
SEQ. ID. NO. 2 (A-chain)
Gly-Ile-Val-Glu-Gln-Cys-Cys-Thr-Ser-Ile-Cys-Ser-Leu-Tyr-Gln-Leu-Glu-Asn-Tyr-Cys-Asn
SEQ. ID. NO. 3 (B-chain)
Phe-Val-Asn-Gln-His-Leu-Cys-Gly-Ser-His-Leu-Val-Glu-Ala-Leu-Tyr-Leu-Val-Cys-Gly-Glu-
Arg-Gly-Phe-Phe-Tyr-Thr-Pro-Lys-Thr

It is envisioned that the insulin analogues of the present invention have affinities for the insulin receptor similar to that of natural insulin but exhibit decreased affinity for Insulin-like Growth Factor Receptor. Insulin or insulin analogue activity may be determined by receptor binding assays as described in more detail herein below. Relative activity may be defined in terms of hormone-receptor dissociation constants (K_{d}), as obtained by curve fitting of competitive displacement assays, or in terms of ED₅₀ values, the concentration of unlabelled insulin or insulin analogue required to displace 50 percent of specifically bound labeled human insulin such as a radioactively-labeled human insulin (such as ¹²⁵I-labeled insulin) or radioactively-labeled high-affinity insulin analog. Alternatively, activity may be expressed simply as a percentage of normal insulin. Affinity for the insulin-like growth factor receptor may also be determined in the same way with displacement from IGFR being measured. In particular, it is desirable for an insulin analogue to have an activity that is 20-200 percent of insulin, such as 25, 50, 110, 120, 130, 140, 150, or 200 percent of normal insulin or more, while having an affinity for IGFR that is less than or equal to 50 percent of normal insulin, such as 10, 20, 30 or 50 percent of normal insulin or less. An insulin analogue can still be useful in the treatment of diabetes even if the in vitro receptor-binding activity is as low as 20% due to slower clearance.

Representative binding studies of insulin analogues known in the art are summarized in Table 1. Because the affinity of insulin for IR (isoform B) is similar to the affinity of IGF-I for IGFR (in each case with K_{d} ca. 0.04 nM), the ratio of respective percent affinities for IR and IGFR (columns 2 and 3), as given in column 4, provides an estimate of the absolute specificity of the insulin analogue. Normalization relative to the specificity of human insulin (row 1) provides an estimate of relative specificity. Relative specificities greater than 1 (less than 1) indicate enhanced (decreased) stringency of receptor binding. In this assay Asp^{B10}-insulin exhibits increased affinity for IGFR, but because affinity for IR is more markedly increased, the relative specificity is greater than 1. Insulin glargine (Lantus®), which contains substitution Asn^{A21}→Gly and a two-residue extension of the B-chain (Arg^{B31} and Arg^{B32}), exhibits increased absolute affinity for IGFR, decreased absolute affinity for IR, and decreased relative stringency of receptor binding. The insulin analogues of the present invention exhibit the oppostive property: decreased absolute affinity for IGFR and increased relative stringency of receptor binding.

TABLE 1
Receptor-Binding Properties of Control Insulin Analogs^{a}

| | relative affinities^{b} | | receptor selectivity | |
|---|---|---|---|---|
| analogue | IR | IGFR | ratio | relative |
| human insulin | 100 | 0.30 ± 0.02 | 333 ± 36 | 1 |
| (ins) | | | | |
| human IGF-I | ND^{c} | 100 | ND | ND |
| lispro-insulin | 92 ± 4 | .27 ± 0.03 | 341 ± 52 | 1.0 ± 0.3 |
| Asp^{B10}-insulin | 336 ± 34 | 0.67 ± 0.07 | 501 ± 100 | 1.5 ± 0.4 |
| DKP-insulin | 236 ± 35 | 1.60 ± 0.24 | 148 ± 49 | 0.4 ± 0.2 |
| Lantus^{e} | 43 ± 7 | 0.92 ± 0.12 | 47 ± 9 | 0.14 ± 0.02 |

^{a}Errors derive from standard errors of the mean.
^{b}The relative affinity of wild-type insulin for IR (column 2) is defined at 100 percent; the relative affinity of IGF-I for IGFR (column 3) is also defined as 100 percent. Respective absolute dissociation constants are similar.

The modifications described herein may be introduced into insulin analogs containing other substitutions and/or additions to the polypeptide sequence of the A or B-chains of insulin. For example, the substitutions described herein may be made in insulin analogues such as Lispro insulin, insulin Aspart, other modified insulins or insulin analogues, or within various pharmaceutical formulations, such as regular insulin, NPH insulin, lente insulin or ultralente insulin, in addition to human insulin. As one example, these modifications may be added to lispro insulin (that is, an insulin analogue also containing the substitutions Lys^{B28}, Pro^{B29}). Lispro insulin is sold under the name Humalog®. Aspart insulin is an insulin analogue that contains an Asp^{B28} substitution and is sold as Novalog®. Both of these analogues are known as fast-acting insulins. The polypeptide sequences of the B-chains of Lispro and Aspart are provided as SEQ. ID. NOS. 4 and 5, respectively.

SEQ. ID. NO. 4 (lispro - B-chain)
Phe-Val-Asn-Gln-His-Leu-Cys-Gly-Ser-His-Leu-Val-Glu-Ala-Leu-Tyr-Leu-Val-Cys-Gly-Glu-
Arg-Gly-Phe-Phe-Tyr-Thr-Lys-Pro-Thr
SEQ. ID. NO. 5 (aspart - B-chain)
Phe-Val-Asn-Gln-His-Leu-Cys-Gly-Ser-His-Leu-Val-Glu-Ala-Leu-Tyr-Leu-Val-Cys-Gly-Glu-
Arg-Gly-Phe-Phe-Tyr-Thr-Asp-Lys-Thr

Another insulin analogue that is known in the art is provided by the substitution of His^{B10} by aspartic acid. Although Asp^{B10}-insulin exhibits favorable pharmaceutical properties, it was also previously associated with increased potential for tumorigenesis in rats, as noted above. The polypeptide sequence of the B-chain of Asp^{B10}-insulin is provided as SEQ. ID. NO. 6.

SEQ. ID. NO. 6 (AspB10 - B-chain)
Phe-Val-Asn-Gln-His-Leu-Cys-Gly-Ser-Asp-Leu-Val-Glu-Ala-Leu-Tyr-Leu-Val-Cys-Gly-Glu-
Arg-Gly-Phe-Phe-Tyr-Thr-Pro-Lys-Thr

Substitutions may also be introduced within an engineered insulin monomer of high activity, designated DKP-insulin, which contains the substitutions Asp^{B10} (D), Lys^{B28} (K) and Pro^{B29} (P). These three substitutions on the surface of the B-chain are believed to impede formation of dimers and hexamers. Use of an engineered monomer circumvents confounding effects of self-assembly on stability assays. The structure of DKP-insulin closely resembles a crystallographic protomer. The sequence of the B-chain polypeptide for DKP-insulin is provided as SEQ. ID. NO. 7.

SEQ. ID. NO. 7 (DKP B-Chain Sequence)
Phe-Val-Asn-Gln-His-Leu-Cys-Gly-Ser-Asp-Leu-Val-Glu-Ala-Leu-Tyr-Leu-Val-Cys-Gly-Glu-
Arg-Gly-Phe-Phe-Tyr-Thr-Lys-Pro-Thr

A-chain analogues of insulin containing novel combinations of A-chain amino-acid substitutions were made by total chemical synthesis of the variant A-chain. Wild-type- and variant B-chains (excepting the DKP B-chain) were obtained from commercial formulations of human insulin or insulin analogues by oxidative sulfitolysis; the DKP B-chain was likewise prepared by total chemical synthesis. The insulin analogues were in each case obtained by insulin chain combination followed by chromatographic purification. In each case the predicted molecular mass was verified by mass spectrometry.

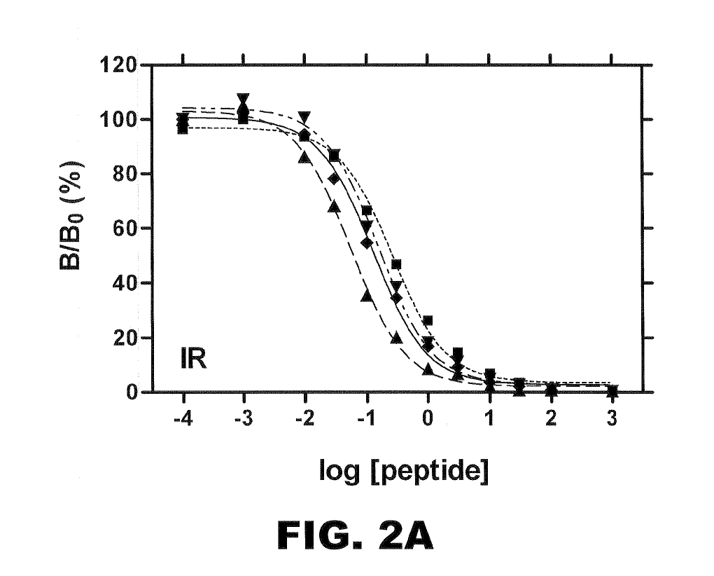

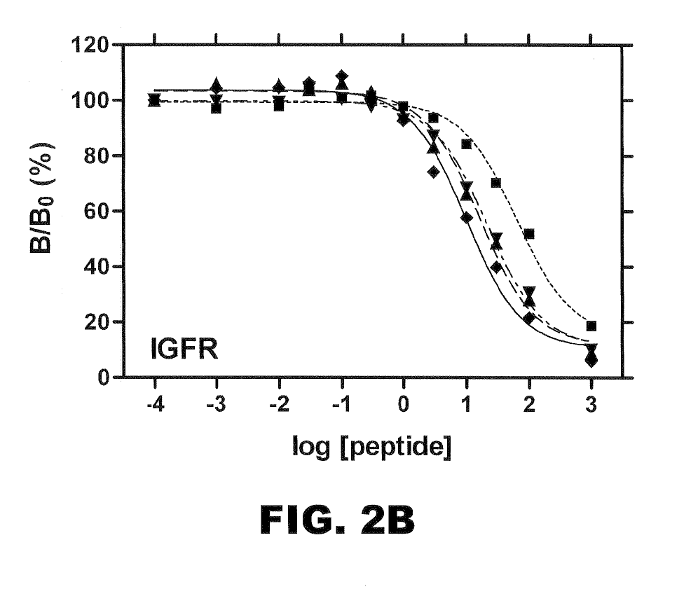

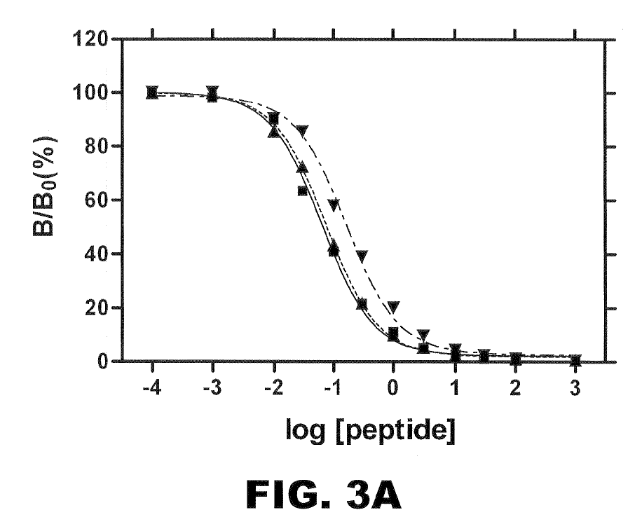

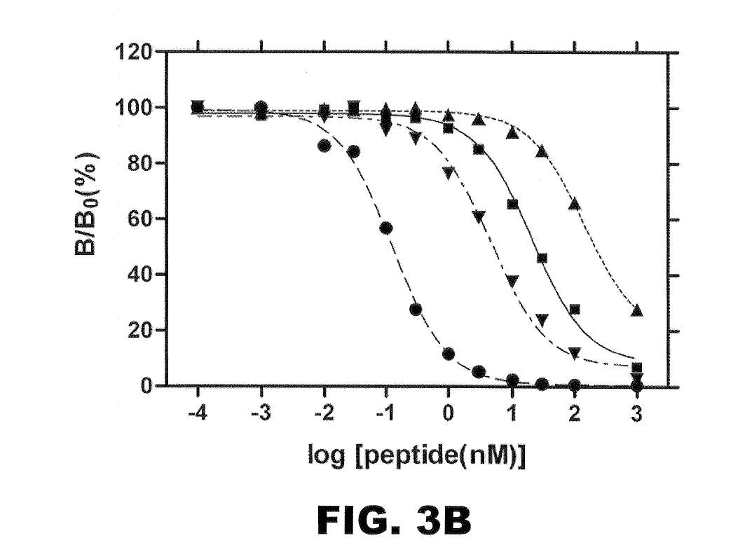

Insulin analogues were synthesized containing the A1, A8 substitutions shown generally as SEQ. ID. NO. 8, in the context of a wild-type human B-chain (SEQ. ID. NO. 3) and DKP B-chain (SEQ. ID. NO. 7). More specifically, the B-chain sequences of these insulin analogues are provided as SEQ. ID. NOS. 9-12. Comparison of the properties of these analogues with human insulin indicates the general effects of A1, A8 substitutions to reduce the affinity of the analogues for IGFR and increase the ratio of affinity for IR versus IGFR (Table 2). Receptor-binding data of insulin analogues containing D-substitutions at position A1 and di-amino-butyric acid at position A8 are provided graphically in FIGS. 2A (binding of insulin analogs to the IR (isoform B)) and 2B (cross-binding of insulin analogs to IGFR).

SEQ. ID. NO. 8 (A1, A8 substitutions)
Xaa₁-Ile-Val-Glu-Gln-Cys-Cys-Xaa₂-Ser-Ile-Cys-Ser-Leu-Tyr-Gln-Leu-Glu-Asn-Tyr-Cys-Asn
Xaa₁ = D-Ala, D-Leu, or D-Asp, Phe
Xaa₂ = His, Lys, Arg, D-orn, Dbu, D-Dpr
SEQ. ID. NO. 9 (Dbu^{A8} substitution)
Phe-Ile-Val-Glu-Gln-Cys-Cys-Xaa-Ser-Ile-Cys-Ser-Leu-Tyr-Gln-Leu-Glu-Asn-Tyr-Cys-Asn
Xaa = Dbu
SEQ. ID. NO. 10 (D-Ala^{A1}, Dbu^{A8} substitutions)
Xaa₁-Ile-Val-Glu-Gln-Cys-Cys-Xaa₂-Ser-Ile-Cys-Ser-Leu-Tyr-Gln-Leu-Glu-Asn-Tyr-Cys-Asn
Xaa₁ = D-Ala
Xaa₂ = Dbu
SEQ. ID. NO. 11 (D-Leu^{A1}, Dbu^{A8} substitutions)
Xaa₁-Ile-Val-Glu-Gln-Cys-Cys-Xaa₂-Ser-Ile-Cys-Ser-Leu-Tyr-Gln-Leu-Glu-Asn-Tyr-Cys-Asn
Xaa₁ = D-Leu
Xaa₂ = Dbu
SEQ. ID. NO. 12 (D-Asp A1, Dbu^{A8} substitutions)
Xaa₁-Ile-Val-Glu-Gln-Cys-Cys-Xaa₂-Ser-Ile-Cys-Ser-Leu-Tyr-Gln-Leu-Glu-Asn-Tyr-Cys-Asn
Xaa₁ = D-Asp
Xaa₂ = Dbu

TABLE 2
Receptor-Binding Properties of A1-A8 Insulin Analogues^{a}

| | relative affinities | | receptor selectivity | |
|---|---|---|---|---|
| analogue | IR | IGFR | ratio | relative |
| human insulin | 100 | 0.3 ± 0.02 | 333 ± 36 | 1 |
| (ins) | | | | |
| human IGF-I | ND^{c} | 100 | ND | ND |
| Dab^{A8}-ins | 91 ± 7 | .13 ± 0.01 | 700 ± 105 | 2.1 ± 0.5 |
| [D-Ala^{A1}, | 48 ± 4 | 0.040 ± 0.002 | 1200 ± 160 | 3.6 ± 0.9 |
| Dbu^{A8}]-ins | | | | |
| [D-Leu^{A1}, | 275 ± 2 | 0.20 ± 0.02 | 1375 ± 150 | 4.1 ± 0.8 |
| Dbu^{A8}]-ins | | | | |
| [D-Asp^{A1}, | 53 ± 4 | 0.050 ± 0.03 | 1060 ± 150 | 3.2 ± 0.8 |
| Dbu^{A8}]-ins | | | | |

Insulin analogues having the A-chain polypeptide sequences of SEQ. ID. NOS. 13-15 were also prepared. Two analogues contain a combination of Arg^{A0} with both an A8 substitution (Arg or His) and substitution of Asn^{A21} by Gly (SEQ. ID. NOS. 13 and 14, respectively). The third lacks Arg^{A0} but retains substitutions His^{A8} and Gly^{A21} (SEQ. ID. NO. 15) to permit their comparison. The analogues were synthesized and tested for activity (Table 3). Each of the three analogues exhibits decreased absolute affinity for IGFR and increased relative stringency. A shift in isoelectric point was verified for two of the analogues (Table 5).

SEQ. ID. NO. 13 (Arg^{A0}, Arg^{A8}, Gly^{A21} substitution)
Arg-Gly-Ile-Val-Glu-Gln-Cys-Cys-Arg-Ser-Ile-Cys-Ser-Leu-Tyr-Gln-Leu-Glu-Asn-Tyr-Cys-
Gly
SEQ. ID. NO. 14 (Arg^{A0}, His^{A8}, Gly^{A21} substitution)
Arg-Gly-Ile-Val-Glu-Gln-Cys-Cys-His-Ser-Ile-Cys-Ser-Leu-Tyr-Gln-Leu-Glu-Asn-Tyr-Cys-Gly
SEQ. ID. NO. 15 (His^{A8}, Gly^{A21} substitution)
Gly-Ile-Val-Glu-Gln-Cys-Cys-His-Ser-Ile-Cys-Ser-Leu-Tyr-Gln-Leu-Glu-Asn-Tyr-Cys-Gly

Insulin analogues having the polypeptide sequences of SEQ. ID. NOS. 16-18 and 20-21 were likewise prepared either with wild type insulin B-chain (SEQ. ID. NO. 3) or with an insulin B-chain polypeptide containing a His^{B1} substitution (SEQ. ID. NO. 19). These analogues contain substitutions at positions A4 and/or A8. The A4-A8 substitutions likewise cause decreased absolute affinity for IGFR and increased relative stringency (Table 4). An upward shift in isoelectric point was verified in representative analogues (Table 5).

SEQ. ID. NO. 16 (Ala^{A4}, His^{A8} substitutions)
Gly-Ile-Val-Ala-Gln-Cys-Cys-His-Ser-Ile-Cys-Ser-Leu-Tyr-Gln-Leu-Glu-Asn-Tyr-Cys-Asn
SEQ. ID. NO. 17 (His^{A4}, His^{A8} substitutions)
Gly-Ile-Val-His-Gln-Cys-Cys-His-Ser-Ile-Cys-Ser-Leu-Tyr-Gln-Leu-Glu-Asn-Tyr-Cys-Asn
SEQ. ID. NO. 18 (Ala^{A4}, His^{A8}, Gly^{A21} substitutions)
Gly-Ile-Val-Ala-Gln-Cys-Cys-His-Ser-Ile-Cys-Ser-Leu-Tyr-Gln-Leu-Glu-Asn-Tyr-Cys-Gly
SEQ. ID. NO. 19 (HisB1 B-chain)
His-Val-Asn-Gln-His-Leu-Cys-Gly-S er-His-Leu-Val-Glu-Ala-Leu-Tyr-Leu-Val-Cys-Gly-Glu-
Arg-Gly-Phe-Phe-Tyr-Thr-Pro-Lys-Thr
SEQ. ID. NO. 20 (His^{A4} A-chain)
Gly-Ile-Val-His-Gln-Cys-Cys-Thr-Ser-Ile-Cys-Ser-Leu-Tyr-Gln-Leu-Glu-Asn-Tyr-Cys-Asn
SEQ. ID. NO. 21 (His^{A8})
Gly-Ile-Val-Glu-Gln-Cys-Cys-His-Ser-Ile-Cys-Ser-Leu-Tyr-Gln-Leu-Glu-Asn-Tyr-Cys-Asn

Receptor-Binding Assays—Relative activity is defined as the ratio of dissociation constants pertaining to the wild-type and variant hormone-receptor complex. Data were corrected for nonspecific binding (amount of radioactivity remaining membrane associated in the presence of 1 μM human insulin). In all assays, the percentage of tracer bound in the absence of competing ligand was less than 15% to avoid ligand-depletion artifacts. Relative affinities of insulin analogues for the isolated insulin holoreceptor (isoform B) were performed using a microtiter plate antibody capture technique as known in the art. Microtiter strip plates (Nunc Maxisorb) were incubated overnight at 4° C. with AU5 IgG (100 μl/well of 40 mg/ml in phosphate-buffered saline). Binding data were analyzed by a two-site sequential model. A corresponding microtiter plate antibody assay using the IGF Type I receptor was employed to assess cross-binding to this homologous receptor.

Relative affinities for insulin analogues containing substitutions at A8 and A21 in the presence or absence of Arg^{A0} are provided in Table 3. Relative affinities for insulin analogues containing substitutions at A4 and A8 are provided in Table 4. Thermodynamic stabilities (as measured by chemical denaturation) and isoelectric points (as measured by isoelectric focusing electrophoresis) of wild-type insulin and selected analogues are provided in Table 5.

TABLE 3
Receptor-Binding Properties of A0-A8-A21 Insulin Analogs^{a}

| | relative affinities | | receptor selectivity | |
|---|---|---|---|---|
| analogue | IR | IGFR | ratio | relative |
| human insulin | 100 | 0.30 ± 0.02 | 333 ± 36 | 1 |
| (ins) | | | | |
| human IGF-I | ND^{c} | 100 | ND | ND |
| Arg^{A0}-Arg^{A8}- | 112 ± 15 | 0.025 ± 0.007 | 4480 ± 1855 | 13.5 ± 7.1 |
| Gly^{A21}-ins | | | | |
| Arg^{A0}-His^{A8}- | 124 ± 22 | 0.038 ± 0.008 | 3263 ± 1158 | 9.8 ± 4.6 |
| Gly^{A21}-ins | | | | |
| His^{A8}-Gly^{A21}- | 184 ± 24 | 0.090 ± 0.016 | 2044 ± 629 | 6.1 ± 2.5 |
| insulin | | | | |

TABLE 4
Receptor-Binding Properties of A4-A8-A21 Insulin Analogs^{a}

| | relative affinities^{b} | | receptor selectivity | |
|---|---|---|---|---|
| analogue | IR | IGFR | ratio | relative |
| human insulin | 100 | 0.30 ± 0.02 | 333 ± 36 | 1 |
| (ins) | | | | |
| human IGF-I | ND^{c} | 100 | ND | ND |
| Ala^{A4}-His^{A8}- | 260 ± 5 | ND | ND | ND |
| insulin | | | | |
| Ala^{A4}-His^{A8}- | 384 ± 64 | 0.35 ± 05 | 1097 ± 340 | 3.3 ± 1.4 |
| DKP-insulin | | | | |
| His^{A4}-His^{A8}- | 90 ± 16 | 0.06 ± 0.01 | 1500 ± 517 | 4.5 ± 2.0 |
| insulin | | | | |
| His^{A4}-His^{A8}- | 29 ± 4 | 0.030 ± 0.007 | 967 ± 359 | 2.9 ± 1.4 |
| Gly^{A21}-ins | | | | |
| His^{A4}-His^{A8}- | 125 ± 18 | 0.05 ± 0.01 | 2500 ± 830 | 7.5 ± 2.5 |
| His^{B1}-ins | | | | |

TABLE 5
Physical Properties of Selected Insulin Analogs

| | | Isoelectric |
|---|---|---|
| analogue | protein stability (ΔGᵤ)^{a} | point (pI) |
| human insulin | 4.4 ± 0.1 | 5.8 |
| Lantus | 2.7 ± 0.1^{b} | 7.2 |
| His^{A4}-insulin | 3.1 ± 0.1 | 6.5 |
| His^{A8}-insulin | 4.9 ± 0.1 | 5.9 |
| His^{A4}-His^{A8}-insulin | 2.3 ± 0.1^{b} | 6.6 |
| His^{A4}-His^{A8}-Gly^{A21}-ins | ND | 6.6 |
| His^{A8}-Gly^{A21}-insulin | 3.1 ± 0.1 | 5.9 |
| Arg^{A0}-His^{A8}-Gly^{A21}-ins | ND | 6.5 |
| His^{B1}-His^{A4}-His^{A8}-ins | ND | 6.7 |
| Ala^{A4}-His^{A8}-DKP-ins | 5.0 ± 0.1 | ND |
| Ala^{A4}-His^{A8}-ins | 4.1 ± 0.1 | ND |
| D-Ala^{A1}-Dbu^{A8}-ins | 3.3 ± 0.1 | ND |
| D-Leu^{A1}-Dbu^{A8}-ins | 3.3 ± 0.1 | ND |
| D-Asp^{A1}-Dbu^{A8}-ins | 3.7 ± 0.1 | ND |

^{a}Values of ΔGᵤ (free energies of unfolding in kcal/mole) were inferred at 4° C. (unless otherwise indicated) from guanidine denaturation studies by application of a two-state model extrapolated to zero denaturant concentration.
^{b}Value was determined at 25° C. and pH 4.0.

The formulation of the present invention provides an intermediate-acting insulin analogue also containing the Lys^{B28} and Pro^{B29} of lispro insulin (Humalog®) that is easily formulated as a clear solution at pH 4 with zinc ions and phenol. Representative binding studies of an insulin analogue containing the lispro and Histidine substitutions at positions A4 and A8 (HisA4, A8 KP-ins) and wild type human insulin (HI) are provided in Table 6 in relation to Human Insulin Receptor Isoform A (HIRA), Human Insulin Receptor Isoform B (HIRA) and Insulin-like Growth Factor Receptor (IGFR). As seen in Table 6, HisA4, A8 KP-ins has a similar affinity for HIRA and HIRB as HI, but a greatly reduced (greater than 4-fold reduced) affinity for IGFR in comparison to HI.

TABLE 6

| | HIRA | | HIRB | | IGFR | |
|---|---|---|---|---|---|---|
| | K_{d} (nM) | SEM | K_{d} (nM) | SEM | K_{d} (nM) | SEM |
| HisA4, A8 KP-ins | 0.016 | 0.003 | 0.033 | 0.005 | 65.3 | 12.3 |
| HI | 0.023 | 0.004 | 0.062 | 0.009 | 13.2 | 2.0 |

FIG. 4 provides a time course of blood glucose levels of diabetic male rats under the following conditions. Male Lewis rats (mean body mass ˜300 g) were rendered diabetic by streptozotocin. Blood glucose concentration following subcutaneous injection were assessed using a clinical glucometer (Hypoguard Advance Micro-Draw meter) in relation to wild-type insulin or buffer alone (16 mg glycerin, 1.6 mg meta-cresol, 0.65 mg phenol, and 3.8 mg sodium phosphate (pH 7.4); Lilly diluent). Rats were injected subcutaneously at time t=0 with 3.44 nmoles of insulin or insulin analogs (˜12-13.7 nmoles) in 100 μl of buffer per rat (for wild-type insulin, this corresponds to 2 IU/kg body weight). Blood was obtained from clipped tip of the tail.

[His^{A4}, His^{A8}]-KP insulin, lispro insulin and insulin glargine were dissolved (like Lantus®) in dilute HCl (pH 4.0) with a molar Zn²⁺:insulin ratio of 5.2:1. The time course of glycemic control for [His^{A4}, His^{A8}]-KP insulin was shorter than for insulin glargine (Lantus®), but longer than for lispro insulin (Humalog®), indicating that this formulation provides an intermediate-acting insulin analogue formulation. Crystals of HisA4, A8 KP-ins have also been obtained (data not shown). While not wishing to be bound by theory, it is believed that the hexamer-destabilizing effects of the lispro substitutions differ from, and are at least partially offset by, the hexamer complex-stabilizing effects of the [His^{A4}, His^{A8}] substitutions, resulting in an intermediate-acting analogue.

Based upon the foregoing disclosure, it should now be apparent that the insulin analogues containing a combination of A-chain substitutions as provided herein will exhibit decreased absolute and relative affinity for the Type I IGR receptor while retaining at least 20% of the affinity of human insulin for the insulin receptor.

We claim:

1. A method treating a patient, the method comprising administering a physiologically effective amount of an insulin analogue or a physiologically acceptable salt thereof to the patient, wherein the insulin analogue or a physiologically acceptable salt thereof contains an insulin A-chain sequence modified at least one position, selected from the group consisting of:
an addition at position A0, wherein the addition is arginine or lysine,
a substitution at position A1, selected from the group consisting of a D-alanine substitution, a D-aspartic acid substitution, and a D-leucine substitution,
an alanine substitution at position A4,
a substitution at position A8 selected from a lysine substitution, an arginine substitution, an ornithine substitution, a D-di-amino-butyric acid substitution, and a D-di-amino-propionic acid substitution, and
a glycine substitution at position A21,
such that the insulin analogue exhibits decreased affinity for the IGF receptor in comparison to wild type insulin of the same species and at least 20% of the affinity of wild-type insulin for the insulin receptor of the same species.

2. The method of claim 1, wherein the insulin analogue or a physiologically acceptable salt thereof has an affinity for IGFR less than two-fold that of wild-type insulin.

3. The method of claim 2, wherein the insulin A-chain sequence is modified at position A1, wherein the modification is selected from the group consisting of a D-alanine substitution, a D-aspartic acid substitution, and a D-leucine substitution, and
wherein the insulin A-chain sequence is further modified at position A8 by substitution with an amino acid selected from histidine, lysine, arginine, ornithine, D-di-amino-butyric acid, and D-di-amino-propionic acid.

4. The method of claim 3, wherein the insulin A-chain sequence is additionally modified at position A21 by substitution with glycine.

5. The method of claim 2, wherein the insulin A-chain sequence is modified at position A4 by alanine and at A8 by substitution with lysine or arginine.

6. The method of claim 5, wherein the analogue is modified at position A21 by substitution with glycine.

7. The method of claim 2, wherein the insulin A-chain sequence is modified at position A0 by an addition of arginine or lysine, and wherein the insulin A-chain sequence is further modified at position A8 by substitution with histidine, lysine, or arginine.

8. The method of claim 7, wherein the analogue also is modified at position A21 by substitution with glycine.

9. The method of claim 1, wherein the insulin analogue or a physiologically acceptable salt thereof additionally comprises an insulin B-chain analogue sequence having an aspartic acid substitution at position B28, or a combination of a lysine substitution at position B28 and a proline substitution at position B29.

10. The method of claim 1, wherein the insulin analogue or a physiologically acceptable salt thereof additionally comprises an insulin B-chain analogue sequence comprising a histidine substitution at position B1.

11. The method of claim 1, wherein the insulin analogue or a physiologically acceptable salt thereof additionally comprises an insulin B-chain analogue sequence extended by a basic amino acid at position B31.

12. The method of claim 1, wherein the insulin analogue or a physiologically acceptable salt thereof additionally comprises an insulin B-chain analogue sequence extended by a basic amino acid at position B31 and a basic amino acid at position B32.

13. The method of claim 1, wherein the insulin analogue or a physiologically acceptable salt thereof is administered to the patient by an implantable pump.

14. The method of claim 1, wherein the insulin analogue or a physiologically acceptable salt thereof is an analogue of a mammalian insulin.

15. The method of claim 14, wherein the insulin analogue or a physiologically acceptable salt thereof is an analogue of human insulin.

16. A composition comprising a vertebrate insulin analogue or a physiologically acceptable salt thereof, comprising a modified insulin A-chain sequence containing at least one modification selected from the group consisting of:
an addition at position A0, wherein the addition is arginine or lysine,
a substitution at position A1, selected from a D-alanine substitution, a D-aspartic acid substitution, and a D-leucine substitution,
an alanine substitution at position A4,
a substitution at position A8 selected from a lysine substitution, an arginine substitution, an ornithine substitution, a D-di-amino-butyric acid substitution, and a D-di-amino-propionic acid substitution, and
a glycine substitution at position A21,
such that the insulin analogue exhibits decreased affinity for the IGF receptor in comparison to wild type insulin of the same species and at least 20% of the affinity of wild-type insulin for the insulin receptor of the same species.

17. The composition of claim 16, wherein the vertebrate insulin analogue additionally comprises an insulin B-chain analogue sequence containing an aspartic acid substitution at position B28 or a combination of a lysine substitution at position B28 and a proline substitution at position B29.

18. The composition of claim 16, wherein the vertebrate insulin analogue additionally comprises an insulin B-chain analogue sequence containing a histidine substitution at position B1.

19. The composition of claim 16, wherein the vertebrate insulin analogue additionally comprises an insulin B-chain analogue sequence extended by a basic amino acid at position B31.

20. The composition of claim 16, wherein the vertebrate insulin analogue additionally comprises an insulin B-chain analogue sequence extended by a basic amino acid at position B31 and a basic amino acid at position B32.

21. The composition of claim 16, wherein the modification is selected from the group consisting of:
an addition at position A0, wherein the addition is arginine or lysine,
a substitution at position A1, selected from a D-alanine substitution, a D-aspartic acid substitution, and a D-leucine substitution,
an alanine substitution at position A4, and
a glycine substitution at position A21,
and wherein the vertebrate insulin analogue additionally contains a histidine substitution at position A8.

22. The composition of claim 16, wherein the modification is selected from the group consisting of:
an addition at position A0, wherein the addition is arginine or lysine,
a substitution at position A1, selected from a D-alanine substitution, a D-aspartic acid substitution, and a D-leucine substitution,
a substitution at position A8 selected from a lysine substitution, an arginine substitution, an ornithine substitution, a D-di-amino-butyric acid substitution, and a D-di-amino-propionic acid substitution, and
a glycine substitution at position A21,
and wherein the vertebrate insulin analogue additionally contains a histidine substitution at position A4.

23. The composition of claim 16, wherein the modification is a substitution at position A1 selected from a D-alanine substitution, a D-aspartic acid substitution, or a D-leucine substitution.

24. The composition of claim 16, wherein the modification is an arginine addition at position A0.

25. A nucleic acid encoding an insulin A-chain polypeptide, wherein the A-chain polypeptide has a sequence selected from the group consisting of SEQ. ID. NOS. 9-18, 20 and 21, and optionally encoding an insulin B-chain polypeptide of SEQ. ID. NO. 19.

26. An expression vector comprising the nucleic acid sequence of claim 25.

27. A host cell transformed with the expression vector of claim 26.

28. A composition comprising a vertebrate insulin analogue or a physiologically acceptable salt thereof, comprising a modified insulin A-chain sequence containing a amino acid substitution at position A1 selected from the group consisting of a D-alanine substitution, a D-aspartic acid substitution, or a D-leucine substitution, and additionally comprising at least one additional modification selected from the group consisting of:
an addition at position A0, wherein the addition is arginine or lysine,
a substitution at position A4, selected from an alanine substitution and a histidine substitution,
a substitution at position A8 selected from the group consisting of a histidine substitution, a lysine substitution, an arginine substitution, an ornithine substitution, a D-di-amino-butyric acid substitution, and a D-di-amino-propionic acid substitution, and
a glycine substitution at position A21.

29. The composition of claim 28, wherein the at least one additional modification is said substitution at position A8 selected from the group consisting of a histidine substitution, a lysine substitution, an arginine substitution, an ornithine substitution, a D-di-amino-butyric acid substitution, or a D-di-amino-propionic acid substitution.
